(19) United States
(12) Patent Application Publication
(10) Pub. No.: US 2014/0130593 A1
(43) Pub. Date: May 15, 2014

(54) PRESSURE MEASUREMENT STRUCTURE

(71) Applicant: INDUSTRIAL TECHNOLOGY RESEARCH INSTITUTE, Hsinchu (TW)

(72) Inventors: Jyun-Kai Ciou, Changhua County (TW); Yan-Rung Lin, Hsinchu (TW); Chang-Ho Liou, Changhua County (TW); Chang-Yi Chen, Hsinchu (TW)

(73) Assignee: INDUSTRIAL TECHNOLOGY RESEARCH INSTITUTE, Hsinchu (TW)

(21) Appl. No.: 13/849,376

(22) Filed: Mar. 22, 2013

(30) Foreign Application Priority Data
Nov. 13, 2012 (TW) 101142301

(51) Int. Cl. A61B 5/103

(52) U.S. Cl. CPC A61B 5/1036
USPC 73/172

(57) ABSTRACT

A pressure measurement structure includes a first substrate, a second substrate, a first electrode layer, a second electrode layer, at least a piezoresistive layer and a wiring layer. The second substrate faces towards the first substrate. The first electrode layer is disposed on the first substrate and faces towards the second substrate. The second electrode layer is disposed on the second substrate and faces towards the first electrode layer. At least a piezoresistive layer is located between the first electrode layer and the second electrode layer. A wiring layer is disposed on the second substrate and back to the first substrate. The wiring layer includes multiple wires. Part of the wires are electrically connected to the first electrode layer. The other part of the wires are electrically connected to the second electrode layer.

### CROSS-REFERENCE TO RELATED APPLICATIONS

This non-provisional application claims priority under 35 U.S.C. §119(a) on Patent Application No(s). 101142301 filed in Taiwan, R.O.C. on Nov. 13, 2012, the entire contents of which are hereby incorporated by reference.

### TECHNICAL FIELD

The disclosure relates to a pressure measurement structure, more particularly to a pressure measurement structure having a high density measuring array.

### RELATED ART

Foot plays a very important role in supporting body weight, reducing stress for lower limbs and related joints, absorbing shock, relieving impact and controlling body balance when contacting the ground. These functions are realized by such tissues as the bones, ligaments and muscles that fit together.

Among common people, at least 80% of them have foot problems Ankle and foot injuries will result in mechanics change to gait, to produce stress on other joints of lower limbs. Thereby, possibly joint lesions may occur. These problems, however, usually can be corrected by appropriate assessment, treatment and cares.

Existing gait assessment is achieved by use of a pressure measurement device. The pressure measurement device is designed to a flake-like measuring board similar to an insole. When used for measurement of foot pressure, it is placed on shoe sole to measure reaction force for patients during walking and moving processes.

To improve measurement accuracy, a pressure measurement device generally incorporates pressure sensing element arrays. The pressure sensing element arrays output the sensing of scanning array pressure by overlapping of electrodes at axis X and axis Y. Nonetheless, the number of arrays will affect the required wiring area. For example, if the number of arrays is 10 times 10, both the axis X and the axis Y require ten groups of electrode wiring provided during design, while the wiring width is limited by process equipment. Hence, given the limited measuring space, partial sensing area is certainly not functioned when wiring area and sensing area are in the same plane. As a result, the accuracy of foot risk assessment project is decreased or the assessment may not be done in particular the assessment for center of pressure (COP). Thereby, misjudgment for COP due to data vacancy of this part is likely to occur.

Consequently, how to improve the accuracy of pressure measurement device under the condition of limited measuring space is an important issue for the research personnel.

### SUMMARY

In an embodiment, the disclosure provides a pressure measurement structure comprising a first substrate, a second substrate, a first electrode layer, a second electrode layer, at least a piezoresistive layer and a wiring layer. The second substrate faces towards the first substrate. The first electrode layer is disposed on the first substrate and faces towards the second substrate. The second electrode layer is disposed on the second substrate and faces towards the first electrode layer. At least a piezoresistive layer is located between the first electrode layer and the second electrode layer. A wiring layer is disposed on the second substrate and back to the first substrate. The wiring layer comprises a plurality of wires. Part of the wires are electrically connected to the first electrode layer. The other part of the wires are electrically connected to the second electrode layer.

### BRIEF DESCRIPTION OF THE DRAWINGS

The present disclosure will become more fully understood from the detailed description given herein below for illustration only, and thus does not limit the present disclosure, wherein:

FIG. 1 is a schematic plan view of the pressure measurement structure according to an embodiment of the disclosure;

FIG. 2 is an exploded view of FIG. 1;

FIG. 3A is an enlarged view of FIG. 1;

FIG. 3B is a simplified sectional view of FIG. 3A, drawn along the section line 3B-3B;

FIG. 4A is a simplified sectional view, drawn along the section line 4A-4A of FIG. 1;

FIG. 4B is a simplified sectional view, drawn along the section line 4B-4B of FIG. 1;

FIG. 4C and FIG. 4D are the partial sectional views of the pressure measurement structure according to another embodiment of the disclosure;

FIG. 5 is the partial sectional view of the pressure measurement structure according to another embodiment of the disclosure; and

FIG. 6 is the partial sectional view of the pressure measurement structure according to still another embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

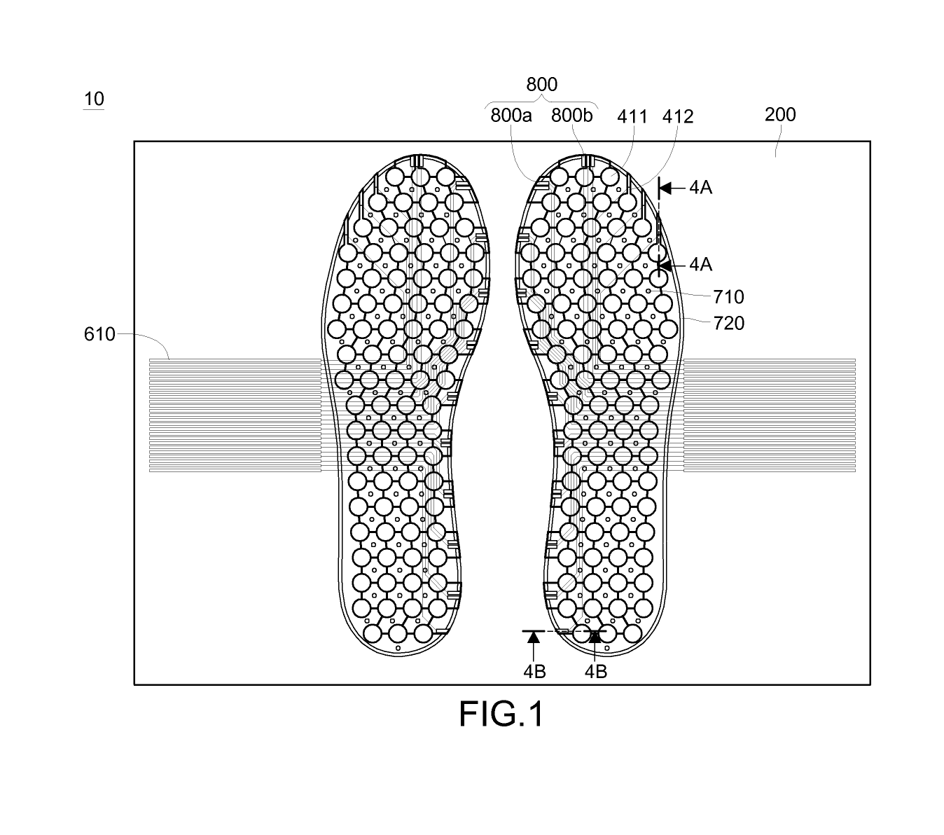

Please refer to FIG. 1. FIG. 1 is a schematic plan view of the pressure measurement structure according to an embodiment of the disclosure. In this embodiment, the pressure measurement structure 10 is configured for measuring the pressure of foot.

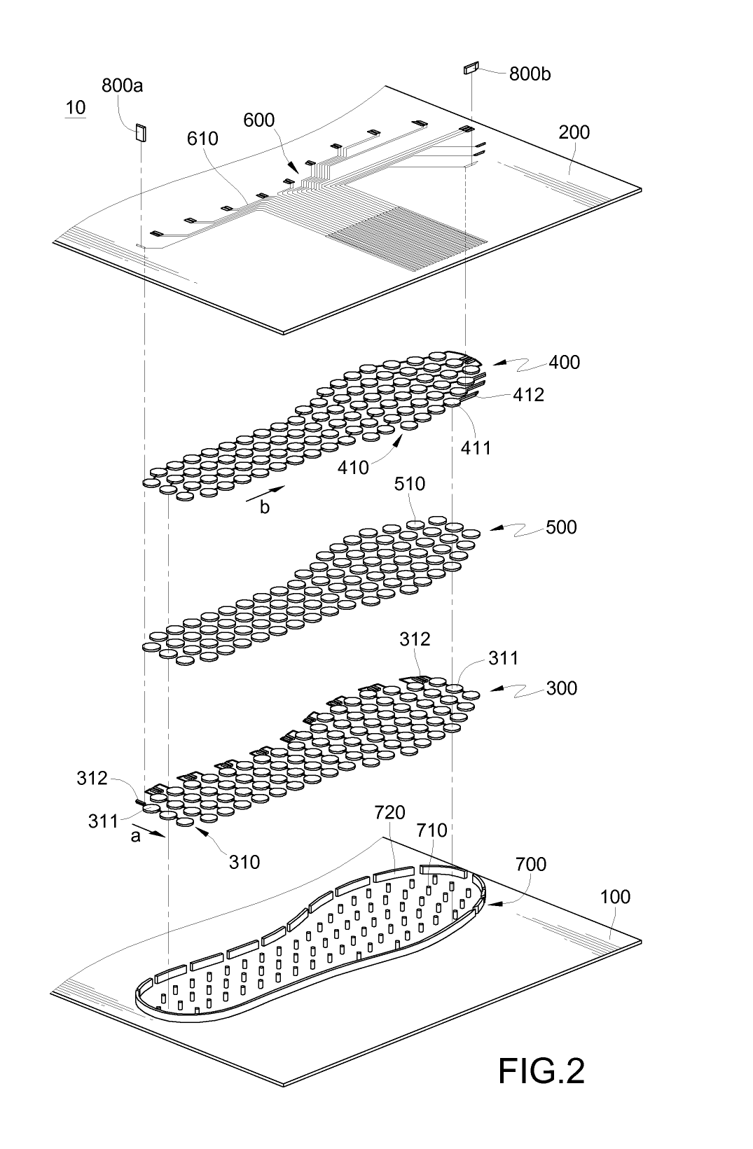

Please refer to FIG. 2, an exploded view of FIG. 1. The pressure measurement structure 10 of this embodiment comprises a first substrate 100, a second substrate 200, a first electrode layer 300, a second electrode layer 400, at least a piezoresistive layer 500 and a wiring layer 600. The pressure measurement structure 10 further comprises an adhesive layer 700. In this embodiment, the first electrode layer 300, the piezoresistive layer 500 and the adhesive layer 700 are actually formed on the first substrate 100 by means of screening printing. The second electrode layer 400 and the wiring layer 600 are actually formed on the second substrate 200 by means of screening printing. The first substrate 100 is made of insulation material.

The first electrode layer 300 is disposed on the first substrate 100. The first electrode layer 300 is made of conductive material. The first electrode layer 300 comprises a plurality of first sensing groups. For example, in FIG. 2, each of the first sensing groups 310 comprises a plurality of first sensing elements 311. The first sensing elements 311 of the first sensing group 310 are electrically connected to each other, while the first sensing elements 311 of different first sensing group 310 are electrically insulated to each other. Moreover, the first sensing elements 311 of the first sensing group 310 are substantially arranged along the first direction (e.g. the direction indicated by the arrow a). The arrangement substantially along the first direction includes both situations of linear arrangement and curve alignment along the first direction.

The piezoresistive layer 500 is located on the first electrode layer 300, and comprises a plurality of piezoresistive elements 510. The piezoresistive elements 510 are arranged in the form of arrays and are electrically connected to the first sensing elements 311 respectively.

The second electrode layer 400 is located above the piezoresistive layer 500. The second electrode layer 400 is made of conductive material. The second electrode layer 400 comprises a plurality of second sensing groups 410. For example, in FIG. 2, each of the second sensing groups 410 comprises a plurality of second sensing elements 411. The second sensing elements 411 of the second sensing group 410 are electrically connected to each other, while the second sensing elements 411 of different second sensing group 410 are electrically insulated to each other. Moreover, the second sensing elements 411 of the second sensing group 410 are substantially arranged along the second direction (e.g. the direction indicated by the arrow b). The arrangement substantially along the second direction includes both situations of linear arrangement and curve alignment along the first direction. The second direction is nearly orthogonal to the first direction.

The second substrate 200 is located on the second electrode layer 400. Furthermore, the second substrate 200 is made of insulation material.

The adhesive layer 700 is disposed on the first substrate 100 or the second substrate 200 (not shown in the figure). The adhesive layer 700 is configured for gluing the first substrate 100 and the second substrate 200. The adhesive layer 700 comprises a plurality of adhesive units 710 and a plurality of adhesive blocks 720. The adhesive units 710 are arranged in the form of arrays and located among the piezoresistive elements 510. The adhesive blocks 720 are arranged at intervals along the circumference of the first electrode layer 300. Specifically, the adhesive layer 700 is configured for gluing the first substrate 100 and the second substrate 200 in the form of lattice arrays. Thereby, air may flow through the space between the first substrate 100 and the second substrate 200. As a result, hysteresis phenomenon due to the air between them that cannot be released may be prevented, leading to affecting the accuracy of the pressure measurement structure 10.

The wiring layer 600 is disposed on the second substrate 200, and the back of the wiring layer 600 faces the first substrate 100. In other words, the wiring layer 600 and the second electrode layer 400 are respectively located on the opposite two sides of the second substrate 200. The wiring layer 600 is made of conductive material, and comprises a plurality of wires 610. Part of the wires 610 are electrically connected to the first electrode layer 300, and the other part of the wires 610 is electrically connected to the second electrode layer 400. The wiring layer 600 is at the different layer respectively from the first electrode layer 300 and the second electrode layer 400, so it does not affect the sensing areas of the first electrode layer 300 and the second electrode layer 400. Additionally, the wiring layer 600 is located at one side of the second substrate, and the circuits of the sensing elements of the first electrode layer 300 and the second electrode layer 400 are centrally connected to the wiring layer 600, so the pressure measurement structure 10 requires only one wiring layer 600, so it can reduce the times of screen printing and the cost and duration for fabrication of the screen board.

Conductive component 800 comprises a plurality of conductive pins 800a, 800b, electrically connected to the wires 610 and the first electrical contacts 312 respectively. These conductive pins 800b are electrically connected to the wires 610 and the second electrical contacts 412 respectively.

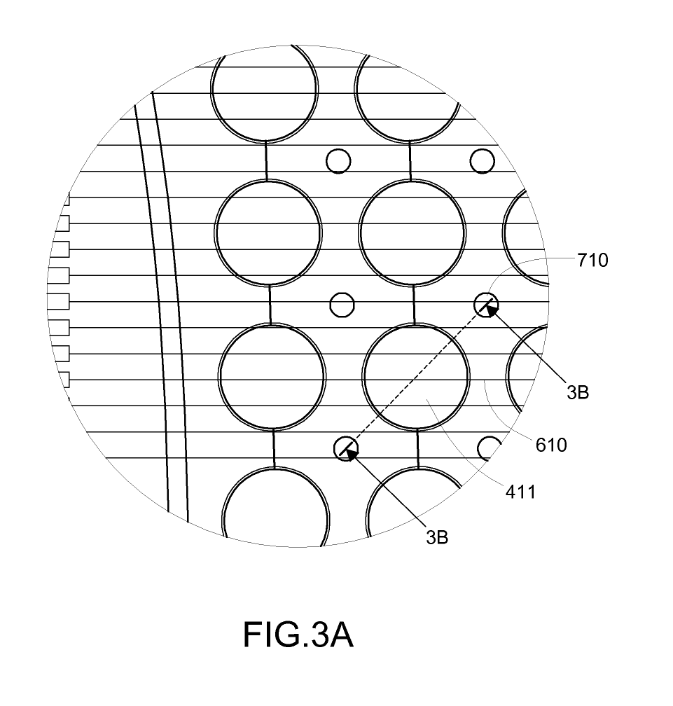

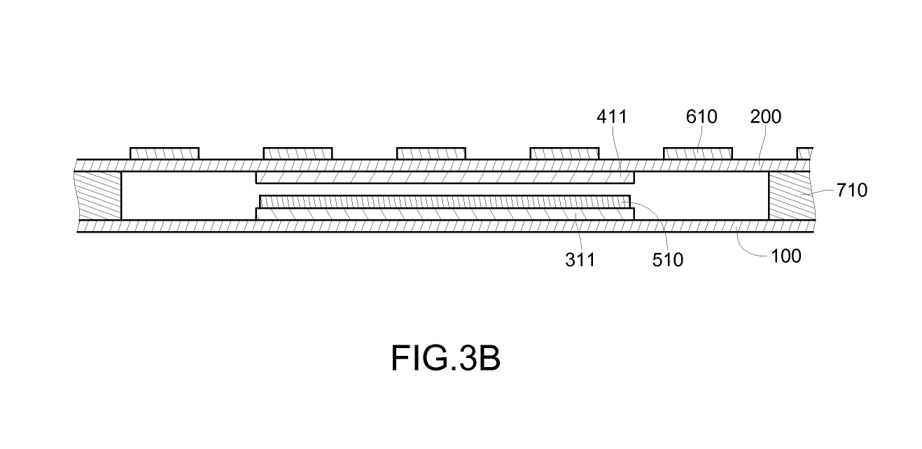

The following is to describe the pressure measurement structure in detail. Please prefer to FIG. 3A and FIG. 3B. FIG. 3A is an enlarged view of FIG. 1. FIG. 3B is a simplified sectional view of FIG. 3A, drawn along the section line 3B-3B. The piezoresistive layer 500 is located on the first electrode layer 300. That is, the piezoresistive elements 510 are respectively located between the first sensing elements 311 and the second sensing elements 411, and overlapped with the first sensing elements 311. The material of the piezoresistive element 510 contains carbon and carbon arrangement in the piezoresistive elements 510 is changed when squeezed, so as to change the resistance value of the piezoresistive element 510. Therefore, the first sensing elements 311 and the second sensing elements 411 are configured for sensing the resistance value changes of the piezoresistive elements 510. For example, when suffered higher pressure, the piezoresistive element 510 has a low resistance value. When suffered lower pressure, the piezoresistive element 510 has a high resistance value. For illustration, take the piezoresistive element 510, the first sensing element 311 and the second sensing element 411 as an example. The piezoresistive element 510 is located between the first sensing element 311 and the second sensing element 411, and form an electrical circuit with the first sensing element 311 and the second sensing element 411. When the piezoresistive element 510 bears pressure, the resistance value of this circuit is changed. Thereby, the corresponding pressure values may be calculated based on these resistance values. Moreover, each first sensing element 311 and each second sensing element 411 are arranged along different directions respectively, so as to form a plane coordinate system. Thus, the pressure values for a plurality of coordinate points is able to be calculated by the pressure measurement structure 10 in accordance with the resistance value between the first sensing element 311 and the second sensing element 411.

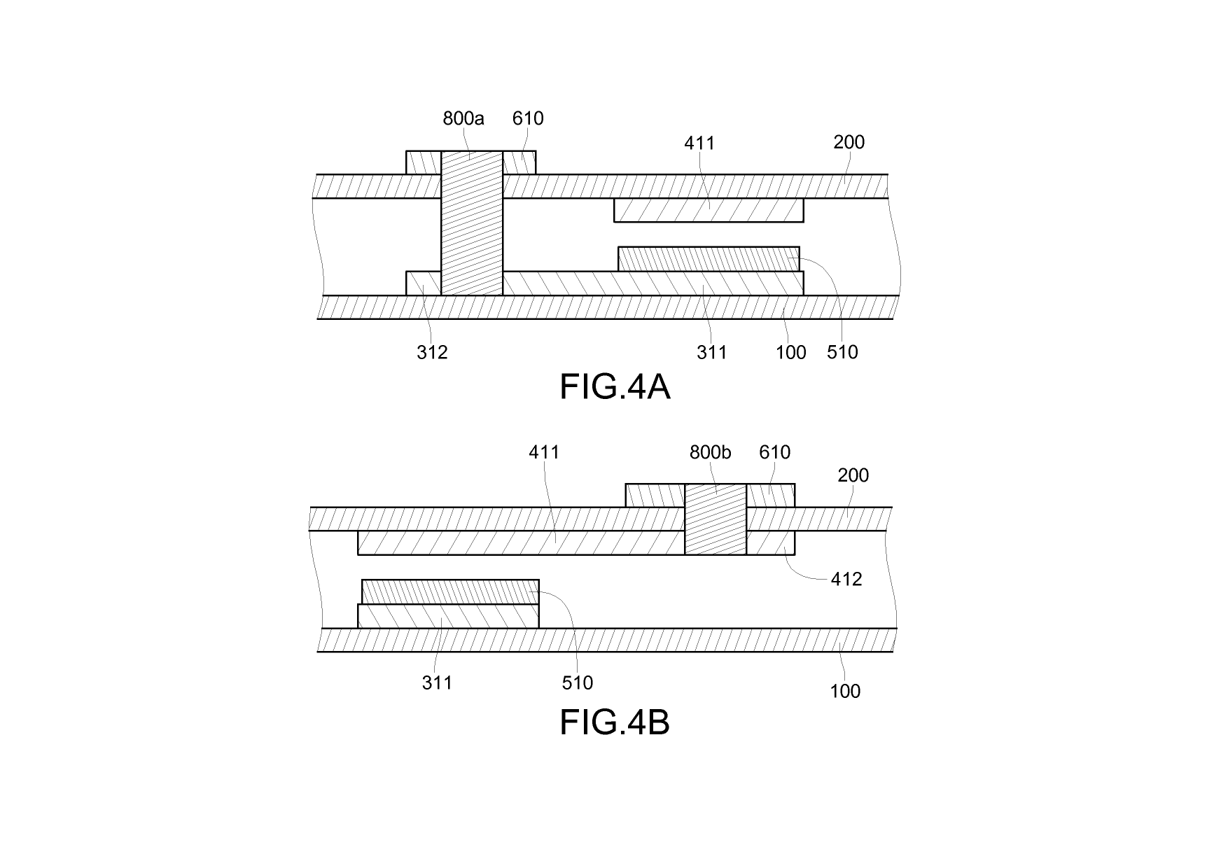

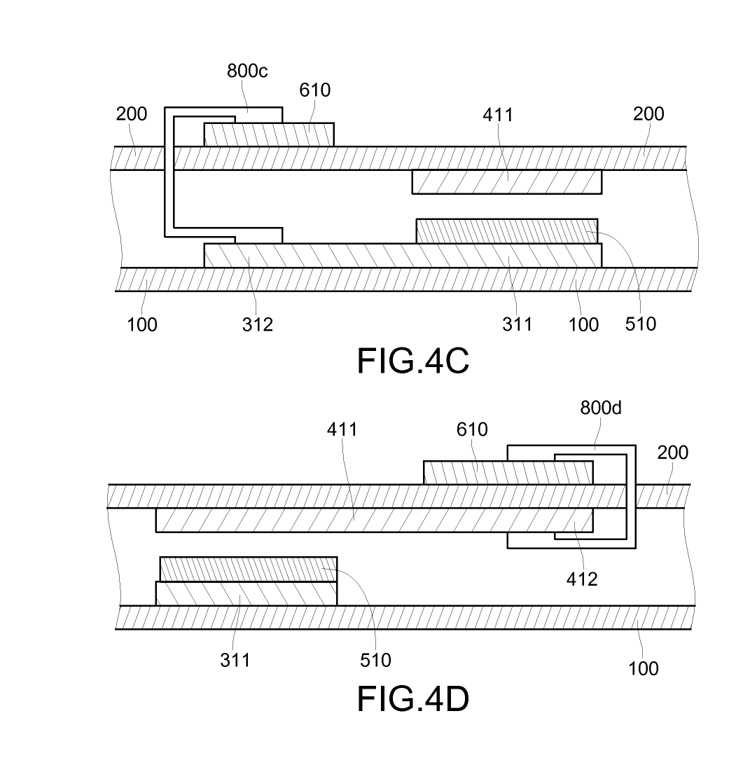

Please refer to FIG. 4A and FIG. 4B. FIG. 4A is a simplified sectional view, drawn along the section line 4A-4A of FIG. 1. FIG. 4B is a simplified sectional view, drawn along the section line 4B-4B of FIG. 1. The pressure measurement structure 10 of this embodiment further comprises a plurality of conductive components 800. The conductive components 800 are made of conductive material. Each of the first sensing groups 310 has a first electrical contact 312. Each of the second sensing groups 410 has a second electrical contact 412. Part of the conductive components 800 are electrically connected to part of wires 610 and each first electrical contact 312. Another part of the conductive components 800 are electrically connected to another part of wires 610 and each second electrical contact 412. As shown in FIG. 4A and FIG. 4B, the conductive pins 800a penetrate through the second substrate 200, and are electrically connected to a wire 610 and the first electrical contact 312 (as shown in FIG. 4A). The conductive pins 800b are electrically connected to another wire 610 and the second electrical contact 412 (as shown in FIG. 4B).

However, the embodiment as drawn in FIG. 4A is not used to limit the variety of conductive components 800. Please refer to FIG. 4C to FIG. 4D. FIG. 4C and FIG. 4D are the partial sectional views of the pressure measurement structure according to another embodiment of the disclosure. In the embodiment as drawn in FIG. 4C, the conductive component 800 comprises a plurality of copper foil tapes 800c and a plurality of 800d. The copper foil tapes 800c are electrically connected to part of wires 610 and each first electrical contact 312 respectively (as shown in FIG. 4C). The copper foil tapes 800d are electrically connected to another part of wires 610 and each second electrical contact 412 (as shown in FIG. 4D).

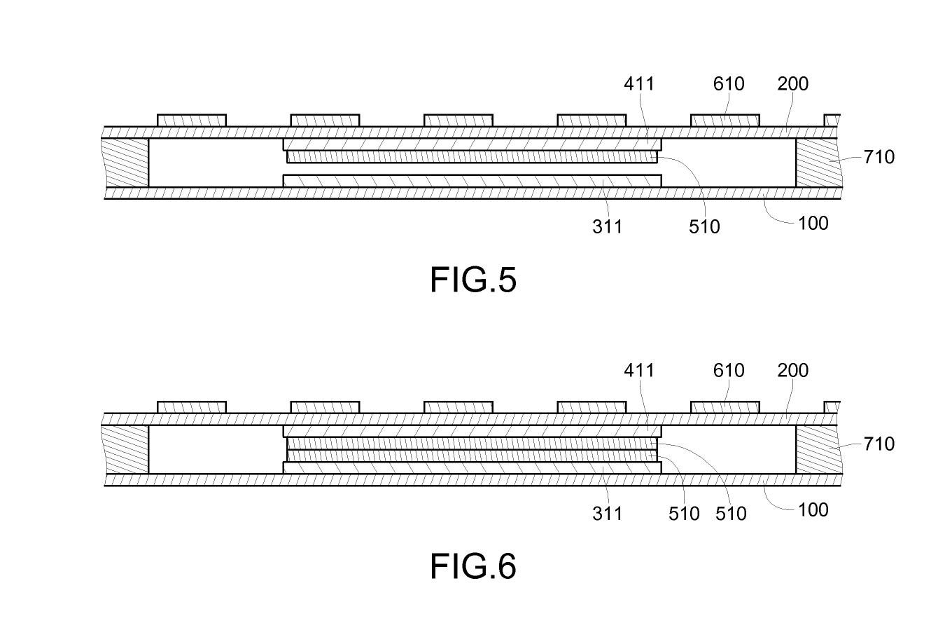

The piezoresistive layer 500 in abovementioned FIG. 3B is disposed on the first electrode layer 300, but the disclosure is not limited thereto. Please refer to FIG. 5, the partial sectional view of the pressure measurement structure according to another embodiment of the disclosure. The piezoresistive element 510 of the piezoresistive layer 500 in this embodiment is assembled on the second sensing element 411 of the second electrode layer 400. Alternatively, refer to FIG. 6. FIG. 6 is the partial sectional view of the pressure measurement structure according to still another embodiment of the disclosure. The quantity of the piezoresistive layers 500 is two, and the two piezoresistive elements 510 of two piezoresistive layers 500 are overlapped on the first sensing element 311 of the first electrode layer 300 and the second sensing element 411 of the second electrode layer 400 respectively.

According to the pressure measurement structure of the disclosure, the wiring layer is separately disposed outside the first electrode layer and the second electrode layer. Thus, the wiring layer does not occupy the sensing area of the first electrode layer and the second electrode layer when sensing elements of the first and second electrode layers are increased. Thereby, the accuracy of pressure measurement device is improved without reducing the sensing area of pressure measurement device.

Furthermore, the wiring layer is assembled on one side of the second substrate, and electrically connected to both the first and the second electrode layers. The pressure measurement structure requires only one wiring layer, so the number of screen boards, the usage amount of printing materials and the process time (that is, times of screen printing) are able to be reduced.

What is claimed is:

1. A pressure measurement structure, comprising:
a first substrate;
a second substrate facing towards the first substrate;
a first electrode layer disposed on the first substrate and facing towards the second substrate;
a second electrode layer disposed on the second substrate and facing towards the first electrode layer;
at least a piezoresistive layer located between the first electrode layer and the second electrode layer; and
a wiring layer disposed on the second substrate and back to the first substrate, wherein the wiring layer comprises a plurality of wires, a part of the wires are electrically connected to the first electrode layer, and another part of the wires are electrically connected to the second electrode layer.

2. The pressure measurement structure according to claim 1, wherein the first electrode layer comprises a plurality of first sensing groups, each of the first sensing groups comprises a plurality of first sensing elements, the first sensing elements of the same first sensing group are electrically connected to each other, the first sensing elements are substantially arranged along a first direction, the second electrode layer comprises a plurality of second sensing groups, each of the second sensing groups comprises a plurality of second sensing elements, the second sensing elements of the same second sensing group are electrically connected to each other, the second sensing elements are substantially arranged along a second direction, the second direction is orthogonal to the first direction.

3. The pressure measurement structure according to claim 2, further comprising a plurality of conductive components, wherein each of the first sensing groups has a first electrical contact, each of the second sensing groups has a second electrical contact, a part of the conductive components are electrically connected to the part of wires and the first electrical contacts, and another part of the conductive components are electrically connected to the another part of wires and the second electrical contacts.

4. The pressure measurement structure according to claim 3, wherein each of the conductive components is a conductive pin, the conductive pins penetrate through the second substrate, the part of the conductive components are electrically connected to the part of the wires and the first electrical contacts, and the another part of the conductive components are electrically connected to the another part of the wires and the second electrical contacts.

5. The pressure measurement structure according to claim 3, wherein each of the conductive components is a copper foil tape, the part of the conductive components are electrically connected to the part of the wires and the first electrical contacts, and the another part of the conductive components are electrically connected to the another part of the wires and the second electrical contacts.

6. The pressure measurement structure according to claim 2, wherein the piezoresistive layer is disposed on the first electrode layer, the piezoresistive layer comprises a plurality of piezoresistive elements, the piezoresistive elements are arranged in several arrays, and the piezoresistive elements are electrically connected to these first sensing elements respectively.

7. The pressure measurement structure according to claim 6, wherein the piezoresistive elements are overlapped with the first sensing elements respectively.

8. The pressure measurement structure according to claim 2, further comprising adhesive layer, wherein the adhesive layer is configured for gluing a first substrate and a second substrate, the adhesive layer comprises a plurality of adhesive units, the adhesive units are arranged in several arrays, and the adhesive units are located among the piezoresistive elements.

9. The pressure measurement structure according to claim 2, wherein the piezoresistive layer is overlapped on the second electrode layer, the piezoresistive layer comprises a plurality of piezoresistive elements, the piezoresistive elements are arranged in several arrays, and the piezoresistive elements are electrically connected to these second sensing elements respectively.

10. The pressure measurement structure according to claim 9, wherein the piezoresistive elements are overlapped with the second sensing elements respectively.

11. The pressure measurement structure according to claim 2, wherein the quantity of piezoresistive layer is two, and the two piezoresistive layers are overlapped on the first electrode layer and the second electrode layer respectively, the two piezoresistive layers comprise a plurality of piezoresistive elements respectively, the piezoresistive elements are arranged in several arrays, and the piezoresistive elements of one of the two piezoresistive layers are electrically connected to the first sensing elements respectively, and the piezoresistive elements of the other of the two piezoresistive layers are electrically connected to the second sensing elements respectively.

12. The pressure measurement structure according to claim 11, wherein the piezoresistive elements of one of the two piezoresistive layers are overlapped with the first sensing elements respectively, and the piezoresistive elements of the other of the two piezoresistive layers are overlapped with the second sensing elements respectively.
